(19) United States
(12) Patent Application Publication
(10) Pub. No.: US 2026/0212464 A1
(43) Pub. Date: Jul. 23, 2026

(54) SYSTEMS AND METHODS FOR IMAGE RECONSTRUCTION

(71) Applicant: SHANGHAI UNITED IMAGING HEALTHCARE CO., LTD., Shanghai (CN)

(72) Inventors: Yihuan LU, Shanghai (CN); Duo ZHANG, Shanghai (CN); Yue LI, Shanghai (CN); Hao LIU, Shanghai (CN); Chen SUN, Shanghai (CN); Enette Mae Constantino REVILLA, Shanghai (CN)

(73) Assignee: SHANGHAI UNITED IMAGING HEALTHCARE CO., LTD., Shanghai (CN)

(21) Appl. No.: 19/570,133

(22) Filed: Mar. 17, 2026

Related U.S. Application Data
(63) Continuation of application No. PCT/CN2024/130202, filed on Nov. 6, 2024.

(30) Foreign Application Priority Data
Nov. 6, 2023 (CN) 202311467045.5
Apr. 2, 2024 (CN) 202410397296.9

(51) Int. Cl. G06T 5/73; A61B 6/00; A61B 6/03; G06T 7/246; G06T 7/33; G06T 12/00

(52) U.S. Cl. CPC G06T 5/73; A61B 6/037; A61B 6/5288; G06T 7/246; G06T 7/33; G06T 12/00; G06T 2207/10104; G06T 2207/20201; G06T 2207/30004; G06T 2210/41

(57) ABSTRACT

The present disclosure provides a system and method for image reconstruction. The method may include obtaining raw positron emission tomography (PET) data of a target object; determining a motion signal of the target object based on the raw PET data of the target object; extracting, based on the motion signal, target PET data from the raw PET data, the target PET data corresponding to a target motion signal, in the motion signal, in which motion change of the target object meets a predetermined change condition; and generating a reconstructed image of the target object by reconstructing the target PET data.

### CROSS-REFERENCE TO RELATED APPLICATIONS

This application is a continuation of International Patent Application NO. PCT/CN2024/130202, filed on Nov. 6, 2024, which claims the priority of Chinese Patent Application No. 202311467045.5, filed on Nov. 6, 2023, and Chinese Patent Application No. 202410397296.9, filed on Apr. 2, 2024, the contents of each of which are hereby incorporated by reference.

### TECHNICAL FIELD

The disclosure generally relates to the field of medical technology, and more particularly relates to systems and methods for image reconstruction.

### BACKGROUND

When using a positron emission tomography (PET) device to scan a target region of a patient, the patient's movements, such as respiratory movements, unconscious body movements, heartbeats, etc., may cause motion artifacts in the scanned image of the patient, which can affect the quality of the captured scanned image. Generally, in order to avoid the impact of motion artifacts on image quality, the main technique is to rely on an external motion detection device to obtain the patient's motion signal during scanning and select PET raw data that is less affected by the motion artifact to generate a reconstructed image. However, the patient's motion signal obtained by the external motion device is easily limited by scanning conditions and cannot fully and accurately obtain motion information. Therefore, it is desired to provide systems and methods for generating a reconstructed image with improved image quality.

### SUMMARY

According to a first aspect of the present disclosure, a system for image reconstruction is provided. The system may include at least one storage device storing executable instructions, and at least one processor in communication with the at least one storage device. When executing the executable instructions, the at least one processor may cause the system to perform one or more of the following operations including: obtaining raw positron emission tomography (PET) data of a target object of a user; determining a motion signal of the target object based on the raw PET data of the target object; extracting, based on the motion signal, target PET data from the raw PET data, the target PET data corresponding to a target motion signal, in the motion signal, in which motion change of the target object meets a predetermined change condition; and generating a reconstructed image of the target object by reconstructing the target PET data.

In some embodiments, the determining a motion signal of the target object based on the raw PET data of the target object comprises: determining a mask of a region of interest (ROI) by processing an image of the target object using a first preset model, and determining, based on a preset algorithm and the mask of the ROI, the motion signal from the raw PET data within the ROI.

In some embodiments, the motion signal includes a respiratory motion signal, and the determining a motion signal of the target object based on the raw PET data of the target object further comprises: determining a correction factor; and correcting the motion signal based on the correction factor.

In some embodiments, the determining a correction factor comprises: obtaining sample raw PET data of multiple sample target objects; for each of the multiple sample target objects, determining sample PET data within a sample ROI of the sample target object based on the sample raw PET data of the sample target object; determining, based on the sample PET data, multiple sample true respiratory amplitudes and multiple sample predicted respiratory amplitudes corresponding to the sample target object; determining a sample correction factor by performing a regression analysis on the multiple sample true respiratory amplitudes and the multiple sample predicted respiratory amplitudes corresponding to the sample target objects; and determining the correction factor based on multiple sample correction factors corresponding to the multiple sample target objects.

In some embodiments, the determining, based on the sample PET data, multiple sample true respiratory amplitudes and multiple sample predicted respiratory amplitudes corresponding to the sample target object comprises: determining centroid movement information as a sample predicted respiratory motion signal by processing the sample PET data using the preset algorithm, wherein an amplitude of the sample predicted respiratory motion signal is represented by a centroid movement distance; determining, by dividing the sample predicted respiratory motion signal, multiple sample predicted sub-signals under multiple respiratory gates; and determining, based on the multiple sample predicted sub-signals, the multiple sample true respiratory amplitudes and the multiple sample predicted respiratory amplitudes corresponding to the sample target object.

In some embodiments, the determining, based on the multiple sample predicted sub-signals, the multiple sample predicted respiratory amplitudes corresponding to the sample target object comprises: for each of the multiple respiratory gates, determining an average respiratory amplitude based on a sample predicted sub-signal corresponding to the respiratory gate; and determining a sample predicted respiratory amplitude corresponding to the respiratory gate by differentiating the average respiratory amplitude corresponding to the respiratory gate with an average respiratory amplitude corresponding to any other respiratory gate of the multiple respiratory gates.

In some embodiments, the determining, based on the multiple sample predicted sub-signals, the multiple sample true respiratory amplitudes corresponding to the sample target object comprises: for each of the multiple respiratory gates, generating a PET sub-image by reconstructing sample target PET sub-data corresponding to a sample predicted sub-signal corresponding to the respiratory gate; determining a first image deformation matrix corresponding to the respiratory gate by registering the PET sub-image with a PET sub-image corresponding to any other respiratory gate of the multiple respiratory gates; and determining, based on a mean value of the first image deformation matrix within the sample ROI, a sample true respiratory amplitude corresponding to the respiratory gate.

In some embodiments, the motion signal includes the respiratory motion signal, and the target motion signal is determined by: selecting, from the respiratory motion signal, a candidate motion signal whose respiratory amplitude is less than or equal to an amplitude threshold; and determining the target motion signal based on the candidate motion signal.

In some embodiments, the determining the target motion signal based on the candidate motion signal comprises: selecting, from the candidate motion signal, multiple consecutive respiratory motion sub-signals in an amplitude direction; and designating a consecutive respiratory motion sub-signal that meets an amplitude span requirement among the multiple consecutive respiratory motion sub-signals as the target motion signal.

In some embodiments, the motion signal includes a body motion signal, and the target motion signal is determined by: determining one or more change point positions by processing the motion signal using a preset detection algorithm; determining multiple body motion sub-signals by dividing, based on the one or more change point positions, the motion signal; and designating a body motion sub-signal that meets a duration condition among the multiple body motion sub-signals as the target motion signal.

In some embodiments, the operations further include: determining a first attenuation coefficient map by processing the reconstructed image using a second preset model; and obtaining an intermediate reconstructed image by correcting, based on the first attenuation coefficient map, the reconstructed image.

In some embodiments, the operations further include: obtaining a second attenuation coefficient map of the target object, the second attenuation coefficient map corresponding to the raw PET data; determining, a second image deformation matrix between the second attenuation coefficient map and the first attenuation coefficient map; and determining a target reconstructed image based on the second image deformation matrix and the intermediate reconstructed image.

According to a second aspect of the present disclosure, a system for image reconstruction is provided. The system includes an obtaining module configured to obtain raw positron emission tomography (PET) data of a target object of a user; a motion signal determination module configured to determine a motion signal of the target object based on the raw PET data of the target object; a target PET data extraction module configured to extract, based on the motion signal, target PET data from the raw PET data, the target PET data corresponding to a target motion signal, in the motion signal, in which motion change of the target object meets a predetermined change condition; and an image generation module configured to generate a reconstructed image of the target object by reconstructing the target PET data.

According to a third aspect of the present disclosure, a method for image reconstruction is provided. The method is implemented on a computing device having at least one processor and at least one storage device, the method comprising: obtaining raw positron emission tomography (PET) data of a target object of a user; determining a motion signal of the target object based on the raw PET data of the target object; extracting, based on the motion signal, target PET data from the raw PET data, the target PET data corresponding to a target motion signal, in the motion signal, in which motion change of the target object meets a predetermined change condition; and generating a reconstructed image of the target object by reconstructing the target PET data.

According to a fourth aspect of the present disclosure, a non-transitory computer readable medium for image reconstruction is provided. The non-transitory computer readable medium comprises at least one set of instructions, wherein when executed by at least one processor of a computing device, the at least one set of instructions direct the at least one processor to perform operations including: obtaining raw positron emission tomography (PET) data of a target object of a user; determining a motion signal of the target object based on the raw PET data of the target object; extracting, based on the motion signal, target PET data from the raw PET data, the target PET data corresponding to a target motion signal, in the motion signal, in which motion change of the target object meets a predetermined change condition; and generating a reconstructed image of the target object by reconstructing the target PET data.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not scaled. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2A is a block diagram illustrating an exemplary processing device according to some embodiments of the present disclosure;

FIG. 2B is a block diagram illustrating an exemplary processing device according to some embodiments of the present disclosure;

FIG. 3 is a flowchart illustrating an exemplary process for image reconstruction according to some embodiments of the present disclosure;

FIG. 4 is a flowchart illustrating an exemplary process for determining a respiratory motion signal of a target object based on raw PET data of the target object according to some embodiments of the present disclosure; and

FIG. 5 is a flowchart illustrating an exemplary process for determining a target reconstructed image according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including” when used in this disclosure, specify the presence of stated features, integers, steps, operations, elements, and/or components, but do not preclude the presence or addition of one or more other features, integers, steps, operations, elements, components, and/or groups thereof.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of exemplary embodiments of the present disclosure.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments in the present disclosure. It is to be expressly understood, the operations of the flowchart may be implemented not in order. Conversely, the operations may be implemented in an inverted order, or simultaneously. Moreover, one or more other operations may be added to the flowcharts. One or more operations may be removed from the flowcharts.

The term “image” in the present disclosure is used to collectively refer to image data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image. The term “region,” “location,” and “area” in the present disclosure may refer to a location of an anatomical structure shown in the image or an actual location of the anatomical structure existing in or on a target subject's body, since the image may indicate the actual location of a certain anatomical structure existing in or on the target subject's body. In some embodiments, an image of an object may be referred to as the object for brevity. Segmentation of an image of an object may be referred to as segmentation of the object. For example, segmentation of an organ refers to segmentation of a region corresponding to the organ in an image.

Provided herein are systems and methods for image reconstruction. For example, the method may include obtaining raw positron emission tomography (PET) data of a target object and determining a motion signal of the target object based on the raw PET data of the target object. The method may further include extracting, based on the motion signal, target PET data from the raw PET data. The target PET data may correspond to a target motion signal, in the motion signal, in which motion change of the target object meets a predetermined change condition. The method may further include generating a reconstructed image of the target object by reconstructing the target PET data.

According to some embodiments of the present disclosure, the motion signal of the target object determined based on the raw PET data of the target object can reflect a motion situation of the target object during the scanning process more accurately, that is, the motion signal is relatively close to a true motion signal of the target object during the scanning process. Thus, based on the motion signal, target PET data that is less affected by the motion of the target object can be extracted from the raw PET data for reconstruction, thereby reducing or eliminating motion artifacts caused by the motion (e.g., respiratory or body motion) during image reconstruction, and improving the quality of reconstructed images.

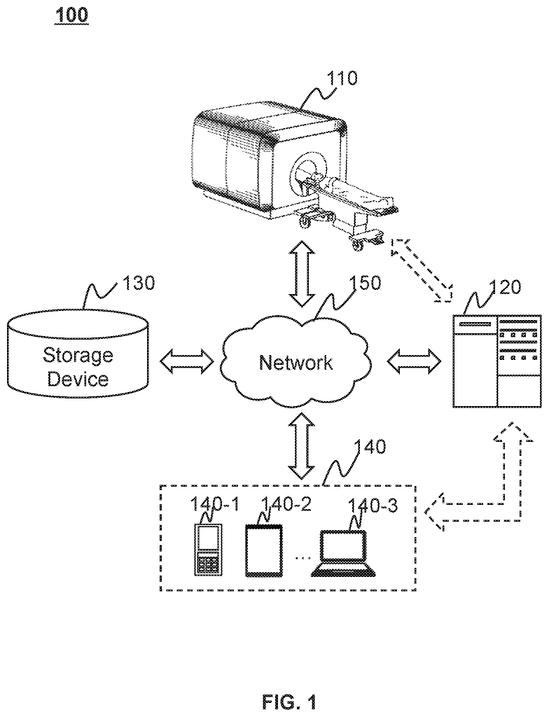

FIG. 1 is a schematic diagram illustrating an exemplary imaging system according to some embodiments of the present disclosure. In some embodiments, the imaging system 100 may be a single-modality system or a multi-modality system. Exemplary single-modality systems may include a positron emission tomography (PET) system, a single-photon emission computed tomography (SPECT) system, etc. Exemplary multi-modality systems may include a magnetic resonance-positron emission tomography (MR-PET) system, a positron emission tomography-computed tomography (PET-CT) system, etc. In some embodiments, the imaging system 100 may include modules and/or components for performing imaging and/or related analysis.

As illustrated in FIG. 1, the imaging system 100 may include an imaging device 110, a processing device 120, a storage device 130, a terminal device 140, and a network 150. The components in the imaging system 100 may be connected to and/or communicate with each other via a wireless connection, a wired connection, or a combination thereof.

The imaging device 110 may be configured to acquire imaging data relating to at least one part of an object. The object may be biological or non-biological. For example, the object may include a patient, a man-made object, etc. As another example, the object may include a specific portion, organ, and/or tissue of the patient. For example, the object may include the head, the neck, the thorax, the heart, the stomach, a blood vessel, soft tissue, a tumor, nodules, or the like, or any combination thereof. In some embodiments, the imaging device 110 may include a positron emission tomography (PET) device, a positron emission tomography-computed tomography (PET-CT) device, a PET-MRI device, or the like, or a combination thereof.

The processing device 120 may process data and/or information relating to image reconstruction to perform one or more functions described in the present disclosure. For example, the processing device 120 may obtain raw PET data of a target object from the imaging device 110 and determine a motion signal of a target object based on raw PET data of the target object. The processing device 120 may further extract target PET data from the raw PET data based on the motion signal and generate a reconstructed image of the target object by reconstructing the target PET data. In some embodiments, the processing device 120 may be a computer, a user console, a single server, a server group, etc. The server group may be centralized or distributed. In some embodiments, the processing device 120 may be local or remote. In some embodiments, the processing device 120 may be implemented on a cloud platform. In some embodiments, the processing device 120 may be implemented on a computing device or the imaging device 110.

The storage device 130 may be configured to store data and/or instructions. The data and/or instructions may be obtained from, for example, the processing device 120, the imaging device 110, and/or any other component of the imaging system 100. In some embodiments, the storage device 130 may store data and/or instructions that the processing device 120 may execute or use to perform exemplary methods described in the present disclosure. In some embodiments, the storage device 130 may include a mass storage device, a removable storage device, a volatile read-and-write memory, a read-only memory (ROM), or the like, or any combination thereof. In some embodiments, the storage device 130 may be implemented on a cloud platform.

The terminal device 140 may be configured to receive information and/or data from the processing device 120, the imaging device 110, and/or the storage device 130 via the network 150. For example, the terminal device 140 may receive an image from the processing device 120. In some embodiments, the terminal device 140 may provide a user interface via which a user may view information and/or input data and/or instructions to the imaging system 100. For example, the user may view, via the user interface, information associated with the imaging device 110. In some embodiments, the terminal device 140 may include a mobile device 140-1, a tablet computer 140-2, a laptop computer 140-3, or the like, or any combination thereof. In some embodiments, the terminal device 140 may include a display that can display information in a human-readable form, such as text, image, audio, video, graph, animation, or the like, or any combination thereof.

The network 150 may facilitate the exchange of information and/or data for the imaging system 100. In some embodiments, one or more components (e.g., the imaging device 110, the processing device 120, the terminal device 140, or the storage device 130) of the imaging system 100 may transmit information and/or data to one or more other components of the imaging system 100 via the network 150. In some embodiments, the network 150 may be any type of wired or wireless network, or combination thereof.

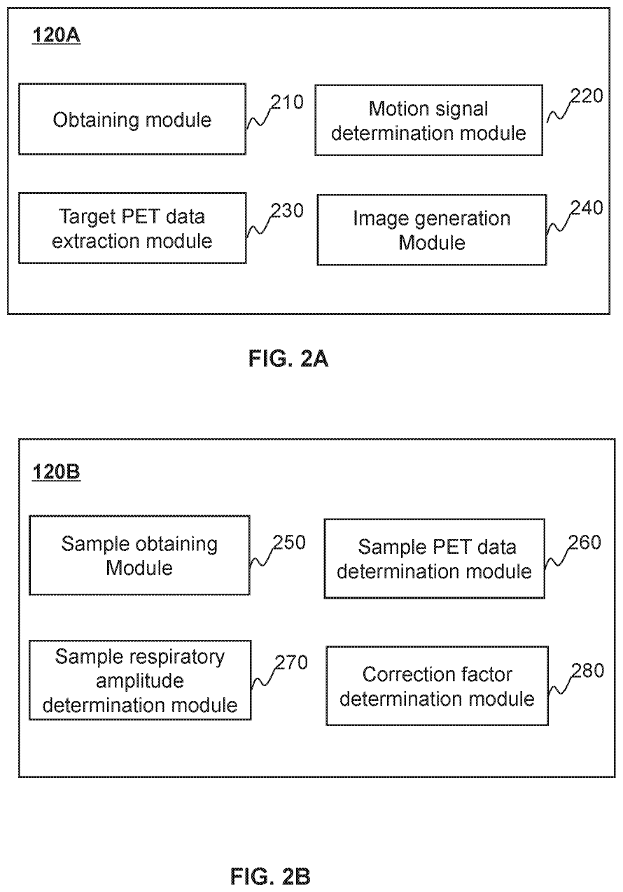

FIG. 2A is a block diagram illustrating an exemplary processing device 120A according to some embodiments of the present disclosure. FIG. 2B is a block diagram illustrating an exemplary processing device 120B according to some embodiments of the present disclosure.

The processing devices 120A and 120B may be exemplary processing devices 120 as described in connection with FIG. 1. In some embodiments, the processing device 120A may be configured to generate a reconstructed image based on target PET data extracted from raw PET data based on a motion signal. The processing device 120B may be configured to determine a correction factor for correcting a respiratory motion signal extracted from the raw PET data. In some embodiments, the processing devices 120A and 120B may be respectively implemented on a processing unit. Alternatively, the processing devices 120A and 120B may be implemented on a same computing unit.

As illustrated in FIG. 2A, the processing device 120A may include an obtaining module 210, a motion signal determination module 220, a target PET data extraction module 230, and an image generation module 240.

The obtaining module 210 may be configured to obtain data and/or information for image reconstruction. For example, the obtaining module 210 may obtain raw positron emission tomography (PET) data of a target object of a user. As another example, the obtaining module 210 may obtain an image of the target object.

The motion signal determination module 220 may be configured to determine a motion signal of the target object based on the raw PET data of the target object.

The target PET data extraction module 230 may be configured to extract, based on the motion signal, target PET data from the raw PET data.

The image generation module 240 may be configured to generate a reconstructed image of the target object by reconstructing the target PET data.

As illustrated in FIG. 2B, the processing device 120B may include a sample obtaining module 250, a sample PET data determination module 260, a sample respiratory amplitude determination module 270, and a correction factor determination module 280.

The sample obtaining module 250 may be configured to may be configured to obtain sample raw PET data of multiple sample target objects.

The sample PET data determination module 260 may be configured to determine sample PET data of a sample ROI of the sample target object based on the sample raw PET data of the sample target object.

The sample respiratory amplitude determination module 270 may be configured to determine, based on the sample PET data, multiple sample true respiratory amplitudes and multiple sample predicted respiratory amplitudes corresponding to the sample target object.

The correction factor determination module 280 may be configured to determine a sample correction factor by performing a regression analysis on the multiple sample true respiratory amplitudes and the multiple sample predicted respiratory amplitudes corresponding to the sample target objects. In some embodiments, the correction factor determination module 280 may determine the correction factor based on multiple sample correction factors corresponding to the multiple sample target objects.

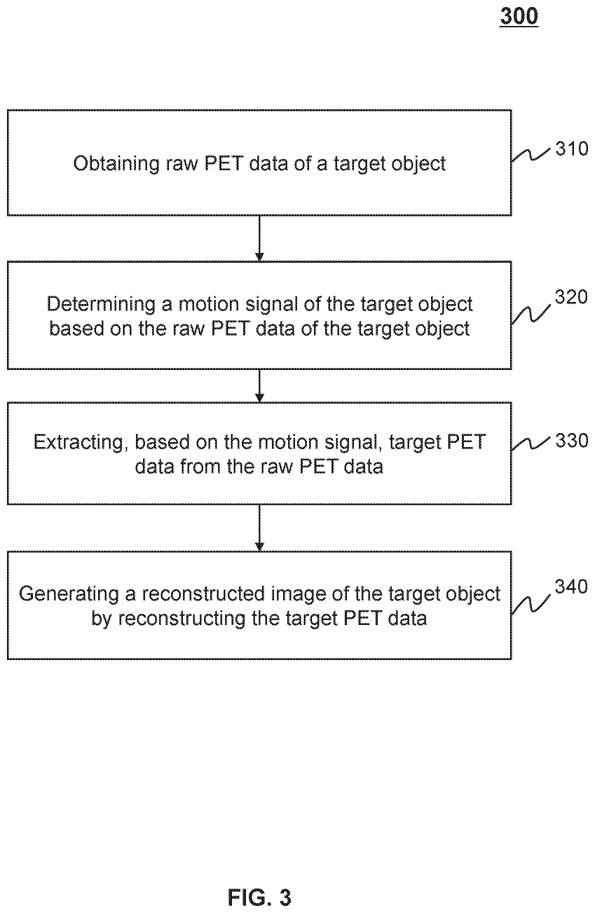

More descriptions of the processing devices 120A and 120B may be found elsewhere of the present disclosure, e.g., FIG. 3 to FIG. 5 and the descriptions thereof.

It should be noted that the above description is merely provided for the purposes of illustration, and is not intended to limit the scope of the present disclosure. Apparently, for persons having ordinary skills in the art, multiple variations and modifications may be conducted under the teachings of the present disclosure. However, those variations and modifications do not depart from the scope of the present disclosure. In some embodiments, the processing device 120A and/or the processing device 120B may share two or more of the modules, and any one of the modules may be divided into two or more units. For instance, the processing devices 120A and 120B may share a same obtaining module; that is, the obtaining module 210 and the sample obtaining module 250 are a same module. In some embodiments, the processing device 120A and/or the processing device 120B may include one or more additional modules, such as a storage module (not shown) for storing data. In some embodiments, the processing device 120A and the processing device 120B may be integrated into one processing device 120.

FIG. 3 is a flowchart illustrating an exemplary process for image reconstruction according to some embodiments of the present disclosure. In some embodiments, process 300 may be executed by the imaging system 100. For example, process 300 may be implemented as a set of instructions (e.g., an application) stored in the storage device 130, and the processing device 120 (e.g., one or more modules in FIG. 2A) may execute the set of instructions and may accordingly be directed to perform the process 300.

In 310, the processing device 120 (e.g., the obtaining module 210 illustrated in FIG. 2A) may obtain raw positron emission tomography (PET) data of a target object.

As used herein, the target object refers to a human body to be scanned. The target object refers to a part of a user that can be affected by the user's movements (e.g., respiratory movements and/or body movements). For example, the target object includes the lungs, the heart, the livers, the spleen, etc.

The raw PET data refers to data obtained by scanning the target object using an imaging device (e.g., the imaging device 110). The imaging device may include a PET device, a PET-CT device, a PET-MRI device, etc., as described elsewhere in the present disclosure (e.g., FIG. 1 and the descriptions thereof). In some embodiments, the raw PET data is obtained from the imaging device directly. Specifically, the processing device may send a scanning command to the imaging device to direct the imaging device to scan the target object, thereby obtaining the raw PET data. In some embodiments, the imaging device transmits acquired raw PET data to the storage device 130 or any other storage device for storage. The processing device 120 may obtain the raw PET data from the storage device 130 or any other storage device.

In 320, the processing device 120 (e.g., the motion signal determination module 220 illustrated in FIG. 2A) may determine a motion signal of the target object based on the raw PET data of the target object.

The motion signal may reflect a motion situation of the target object during a scanning process of collecting the raw PET data. In some embodiments, the motion signal of the target object includes a respiratory motion signal, a body motion signal, a heartbeat signal, etc. In some embodiments, the body motion signal is caused by unconscious movements (e.g., shaking) of body parts such as the body, head, limbs, etc.

In some embodiments, the processing device 120 may determine the motion signal using a motion signal prediction model. Specifically, the processing device 120 may input the raw PET data into the motion signal prediction model to determine the motion signal. The motion signal prediction model may be trained based on a plurality of groups of training data. Each group of training data may include sample raw PET data, and during the training, the label sample motion signal.

In some embodiments, the processing device 120 extracts the motion signal of the target object from a region of interest (ROI) that is affected by movements (e.g., respiratory and/or body movements) of the target object. The ROI may be a part or all of an image of the target object. Merely by way of example, the image includes a whole human body. The ROI includes a human body cavity that is easily affected by the respiratory movement of a human body (e.g., a patient), such as the pleural, the abdominal cavity, the pelvic cavity, etc.

In some embodiments, the processing device 120 first obtains the image of the target object, for example, by scanning the target object using an imaging device or from a storage device.

In some embodiments, the image includes a CT image, a PET image, an MRI image, a PET direct back projection image (histoimage), an integrated multi-channel image of PET/CT/MRI images, etc., corresponding to the raw PET data. For ease of illustration, a CT image is taken as an example of the image in the present disclosure.

The processing device 120 may determine a mask of the ROI (also referred to as an ROI mask) by processing the image of the target object using a first preset model. Specifically, the processing device 120 may input the image into the first preset model to determine the ROI mask. The first preset model may be trained based on a plurality of groups of training data. Each group of training data may include sample images, and during the training, the label is manually segmented regions affected by movements of the user. In some embodiments, if the image is an integrated multi-channel image of PET/CT/MRI images, the first preset model may include multiple input channels each of which corresponds to one type of image. The processing device 120 may input each image in the integrated multi-channel image into the corresponding input channel of the first preset model to determine the ROI mask.

In some embodiments, the first preset model includes a machine learning model, for example, a convolutional neural network (CNN) model, a generative adversarial network (GAN) model, a fully convolutional neural network (FCN) (e.g., a U-Net, a V-Net), a recurrent neural network (RNN), a diffusion model, a transformer, or the like, or any combination thereof.

Further, the processing device 120 may apply the ROI mask to a raw PET image corresponding to the raw PET data to determine raw PET data within the ROI. The processing device 120 may determine, based on a preset algorithm, the motion signal from the raw PET data within the ROI. In some embodiments, the preset algorithm may include a centroid-based respiratory signal analysis technique or a centroid-based body motion signal analysis technique.

Merely by way of example, the processing device 120 may determine a centroid of the ROI. For example, the image may be a point cloud image composed of points. The processing device 120 may calculate the centroid based on all points in the ROI. Since the ROI is affected by the movements of the target object, the centroid of the ROI may fluctuate in position with the movements, and this fluctuation can represent the motion signal of the target object. The processing device 120 may determine centroid movement information of the centroid by processing the raw PET data e.g., using the preset algorithm. The processing device 120 may extract the motion signal based on the centroid movement information of the centroid using, e.g., a spectral filtering technique. An amplitude of the motion signal is represented by a centroid movement distance of the centroid.

Specifically, the processing device 120 may determine a centroid movement distance (also referred to as a movement distance of the centroid) in a true movement direction (i.e., a direction in which the centroid actually moves) of the centroid as an amplitude of the motion signal. The processing device 120 may further determine a plurality of centroid movement distances at different time points, which constute the motion signal of the target object. For example, if the motion signal includes the body motion signal, the body motion signal is a multidimensional signal representing the variation of movement distances of the centroid in different directions.

In some embodiments, the processing device 120 may determine a projection of the centroid movement distance on the X-axis, Y-axis, or Z-axis of a three-dimensional cartesian coordinate system as the amplitude of the motion signal. The processing device 120 may further determine a plurality of projections of the plurality of centroid movement distances at different time points, which constute the motion signal of the target object.

In some embodiments, the processing device 120 may determine a motion signal in a target direction as the motion signal of the target object. For example, when the motion signal includes the respiratory motion signal, the processing device 120 may perform Fourier transform on a respiratory motion signal in any direction to obtain a frequency domain signal. The processing device 120 may select a segment of the frequency domain signal in a low-frequency range as a respiratory motion signal frequency band, and determine the signal greater than the respiratory motion signal frequency band as a noise frequency band. The processing device 120 may further calculate a ratio of the respiratory motion signal frequency band to the noise frequency band to obtain a signal-to-noise ratio. The processing device 120 may take a direction with the highest signal-to-noise ratio as a direction with the best respiratory motion signal quality, that is, take the motion signal in the direction with the highest signal-to-noise ratio as the motion signal in the target direction. As another example, the target direction is designated by the user or operator.

In some embodiments, the target direction is any direction of the three-dimensional cartesian coordinate system. In some embodiments, the target direction is a direction fused based on at least two directions. For example, the target direction is a direction determined by fusing the X-axis direction and the Y-axis direction of the three-dimensional cartesian coordinate system. The processing device 120 may determine a fusion of motion signals in the X-axis direction and the Y-axis direction as the motion signal in the target direction.

According to some embodiments, by extracting the motion signal from the raw PET data within the ROI, the obtained motion signal can be better reflect the true motion situation of the user.

In some embodiments, if the motion signal includes the respiratory motion signal, since the respiratory motion signal is extracted directly from the raw PET data, the extracted respiratory motion signal can not reflect the true respiratory amplitude of the respiratory motion signal (i.e., a true respiratory motion signal) of the user. Thus, in order to improve the accuracy of the extracted motion signal, that is, to make the extracted motion signal closer to the true respiratory motion signal, the processing device 120 may further determine a correction factor (also referred to as a respiratory correction factor), and correct the respiratory motion signal to generate a corrected respiratory motion signal based on the correction factor. Specifically, the processing device 120 may correct the motion signal by calculating a product of the correction factor and an amplitude of each point on the motion signal.

As used herein, the correction factor is determined based on sample respiratory motion signals obtained using two different manners. The the two different manners at least include a first manner of extracting the sample respiratory motion signals from sample raw PET data, and a second manner of extracting the sample respiratory motion signals from reconstructed PET sub-images corresponding to sample PET sub-data. The sample PET sub-data is generated by gating the sample raw PET data. In some embodiments, the correction factor represents a mapping relationship between a ture respiratory amplitude (i.e., an amplitude of a respiratory motion signal that is close to the true respiratory motion signal of the target object) and a predicted respiratory amplitude (i.e., the amplitude of the respiratory motion signal extracted from the raw PET data). The processing device 120 may transform the respiratory motion signal extracted from the raw PET data to a true respiratory motion signal using the correction factor. Specifically, the processing device 120 may determine the true respiratory motion signal by multiplying the amplitude of the respiratory motion signal extracted from the raw PET data by the correction factor.

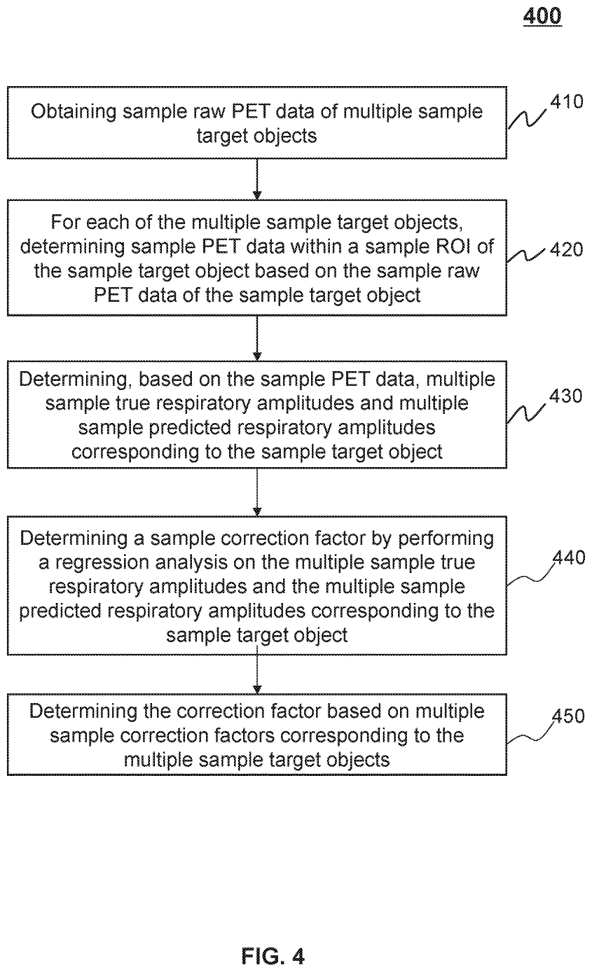

In some embodiments, the processing device 120 may determine the correction factor based on sample raw PET data of multiple sample target objects. For example, for each of the multiple sample target objects, the processing device 120 may determine sample PET data within a sample ROI of the sample target object based on the sample raw PET data of the sample target object. The processing device 120 may determine, based on the sample PET data, multiple sample true respiratory amplitudes and multiple sample predicted respiratory amplitudes corresponding to the sample target object. The processing device 120 may determine a sample correction factor by performing a regression analysis on the multiple sample true respiratory amplitudes and the multiple sample predicted respiratory amplitudes corresponding to the sample target objects. The processing device 120 may determine the correction factor based on multiple sample correction factors corresponding to the multiple sample target objects. More descriptions for determining the correction factor may be found in FIG. 4 and the descriptions thereof.

In 330, the processing device 120 (e.g., the target PET data extraction module 230 illustrated in FIG. 2A) may extract, based on the motion signal, target PET data from the raw PET data. The target PET data corresponds to a target motion signal, in the motion signal, in which motion change of the target object meets a predetermined change condition.

In some embodiments, when the motion signal includes the respiratory motion signal, the predetermined change condition is that a respiratory amplitude of a respiratory motion sub-signal (i.e., a portion of the respiratory motion signal) is less than an amplitude threshold. In such cases, the obtained respiratory motion sub-signal is a stable respiratory motion signal segment. In some embodiments, the amplitude threshold is also referred to as a maximum tolerable respiratory amplitude (i.e., a respiratory amplitude corresponding to the maximum allowance artifacts in the reconstructed image caused by the respiratory amplitude). In some embodiments, the maximum tolerable respiratory amplitude is set according to a default setting of the imaging system 100 or preset by a user or operator via the terminal device 140.

In some embodiments, the amplitude threshold is determined based on historical respiratory motion signals of multiple historical target objects (e.g., patients). For example, the processing device 120 determines an initial tolerable respiratory amplitude based on the historical respiratory motion signals. The processing device 120 may directly determine the initial tolerable respiratory amplitude as the amplitude threshold. Alternatively, the processing device 120 may also determine the amplitude threshold based on the initial tolerable respiratory amplitude. For example, the processing device 120 may determine a certain proportion (e.g., 50%, 60%, 80%, 90%, etc.) of the initial tolerable respiratory amplitude as the amplitude threshold.

In some embodiments, the initial tolerable respiratory amplitude may be determined based on historical data (including historical respiratory motion signals, historical amplitude thresholds, etc.) of the multiple historical target objects relating to determining corresponding historical amplitude thresholds. For example, the processing device 120 may determine, based on the historical data, a relationship between a historical amplitude threshold and a historical maximum respiratory amplitude, a relationship between the historical amplitude threshold and a historical minimum respiratory amplitude, and a relationship between the historical amplitude threshold and a historical respiratory amplitude mean value, respectively. The processing device 120 may average history amplitude thresholds, historical maximum respiratory amplitudes, historical minimum respiratory amplitudes, and historical respiratory amplitude mean values, respectively. As a result, a first target relationship between the average history amplitude threshold and the average historical maximum respiratory amplitude, a second target relationship between the average history amplitude threshold and the average historical minimum respiratory amplitude, and a third target relationship between the average history amplitude threshold and the average historical respiratory amplitude mean value are determined respectively. For example, the first target relationship is a proportional relationship between the average history amplitude threshold and the average historical maximum respiratory amplitude. The second target relationship is a proportional relationship between the average history amplitude threshold and the average historical minimum respiratory amplitude. The third target relationship is a proportional relationship between the average history amplitude threshold and the average historical respiratory amplitude mean value.

Further, the processing device 120 may determine the initial tolerable respiratory amplitude based on the target relationships (including the first target relationship, the second target relationship, and the third target relationship). For example, the processing device 120 may determine the average historical maximum respiratory amplitude, the average historical minimum respiratory amplitude, and the average historical respiratory amplitude mean value in the target relationships as a maximum respiratory amplitude, a minimum respiratory amplitude, and a respiratory amplitude mean value of the current target object, respectively. The processing device 120 may weigh the maximum respiratory amplitude, the minimum respiratory amplitude, and the respiratory amplitude mean value of the current target object to determine the initial tolerable respiratory amplitude.

In some embodiments, a weight corresponding to the respiratory amplitude mean value may be greater than each of weights corresponding to the maximum respiratory amplitude and the minimum respiratory amplitude of the current target object. In some embodiments, weights of the maximum respiratory amplitude, the minimum respiratory amplitude, and the respiratory amplitude mean value of the current target object may be related to a difference between the maximum respiratory amplitude and the minimum respiratory amplitude of the current target object. The greater the difference is, the closer the three weights of the maximum respiratory amplitude, the minimum respiratory amplitude, and the respiratory amplitude mean value of the current target object may be.

In some embodiments, the processing device 120 may determine the initial tolerable respiratory amplitude using a machine learning model. The machine learning model may be trained based on a plurality of groups of training data. Each group of training data includes sample historical data relating to determining a corresponding sample historical amplitude threshold, and during the training, the label is the corresponding sample historical amplitude threshold.

After the amplitude threshold is determined, the processing device 120 may select, from the respiratory motion signal (or the corrected respiratory motion signal), a candidate motion signal (or a candidate respiratory motion signal) whose respiratory amplitude is less than or equal to the amplitude threshold. For example, the processing device 120 may select the candidate respiratory motion signal starting from a positon of the respiratory motion singal (or the corrected respiratory motion signal) whose corresponding respiratory amplitude is 0, or any other positon of the respiratory motion singal (or the corrected respiratory motion signal). As another example, the processing device 120 may select the candidate respiratory motion signal based on a histogram of the respiratory motion signal (or the corrected respiratory motion signal).

The processing device 120 may determine the target motion signal (or a target respiratory motion signal) based on the candidate respiratory motion signal. Specifically, the processing device 120 may select, from the candidate respiratory motion signal, multiple consecutive respiratory motion sub-signals in an amplitude direction of the respiratory motion signal. As used herein, the amplitude direction refers to a numerically continuous interval. That is, amplitudes of the selected multiple consecutive respiratory motion sub-signals are numerically continuous. Merely by way of example, the processing device 120 may determine the multiple consecutive respiratory motion sub-signals by taking a preset proportion of the candidate respiratory motion signal. In some embodiments, the preset proportion may be determined based on an amount of data required for the target motion signal. In some embodiments, the preset proportion may be set according to a default setting of the imaging system 100 or preset by a user or operator via the terminal device 140. The processing device 120 may designate a target consecutive respiratory motion sub-signal that meets an amplitude span requirement among the multiple consecutive respiratory motion sub-signals as the target respiratory motion signal.

As used herein, the amplitude span requirement refers that an amplitude span of a consecutive respiratory motion sub-signal (i.e., a difference between a smallest amplitude and a largest amplitude in the consecutive respiratory motion sub-signal) is less than an amplitude span threshold. Merely by way of example, the amplitude span requirement is that an amplitude span of a consecutive respiratory motion sub-signal is the smallest. That is to say, the target respiratory motion signal is the target consecutive respiratory motion sub-signal with the smallest amplitude span. In other words, the target respiratory motion signal is the target consecutive respiratory motion sub-signal that is least affected by the respiratory movement of the target object among the multiple consecutive respiratory motion sub-signals.

In some embodiments, the processing device 120 may select the target consecutive respiratory motion sub-signal (i.e., the target respiratory motion signal) based on a histogram of the candidate respiratory motion signal. In some embodiments, the processing device 120 may select the target consecutive respiratory motion sub-signal (i.e., the target respiratory motion signal) that meets the amplitude span requirement using a slope of a respiratory curve representing the variation of respiratory amplitude over time. The smaller the slope of the respiratory curve is, the smaller the amplitude span may be.

According to some embodiments, by further screening a consecutive respiratory motion sub-signal that meets the amplitude span requirement (e.g., with the smallest amplitude span), the selected respiratory motion sub-signal can be more stable, thereby reducing motion blur artifacts caused by respiratory motion in reconstructed images.

In some embodiments, since the more data in the consecutive respiratory motion sub-signal, the richer the information in the corresponding raw PET data, and the higher the quality of the reconstructed image, the processing device 120 may determine a consecutive respiratory motion sub-signal that has the most data among the multiple consecutive respiratory motion sub-signals as the target respiratory motion signal.

In some embodiments, the processing device 120 may determine a consecutive respiratory motion sub-signal that has a minimum respiratory amplitude as the target respiratory motion signal.

In some embodiments, when the motion signal includes the body motion signal, the predetermined change condition is that a body motion sub-signal that meets a duration condition. In some embodiments, the processing device 120 may divide the body motion signal into multiple body motion sub-signals based on one or more changing point positions in the body motion signal. As used herein, a change point position refers to a position in the body motion signal where the corresponding amplitude exceeds an amplitude threshold. The processing device 120 may determine the body motion signal segment between two adjacent change point positions as a body motion sub-signal.

In some embodiments, the one or more change point positions may be determined by processing the body motion signal using a preset detection algorithm. Exemplary preset detection algorithms may include a Bayesian online change point detection algorithm, a piecewise linear regression algorithm, a pruning precise linear time algorithm, a statistical-based detection algorithm, a clustering-based detection algorithm, a filtering-based signal extraction algorithm, a deep learning-based detection algorithm, or the like, or any combination thereof.

In some embodiments, the body motion signal may be a multidimensional signal representing the variation of movement distances of the centroid in different directions. In such cases, the processing device 120 may regard the multidimensional signal (i.e., the body motion signal) as a whole signal and further process the whole signal using the preset detection algorithm to determine the one or more change point positions. In some embodiments, the processing device 120 may determine change point positons in each single direction, respectively. The processing device 120 may determine a gather of the change points in signals of the different directions in the body motion signal as the one or more change point positions of the body motion signal.

In some embodiments, the processing device 120 may also determine the one or more change point positions in the body motion signal using a change point positon determination model. The change point positon determination model may be a machine learning model trained based on a plurality of groups of training data. Each group of training data includes sample body motion signal, and during the training, the label is the corresponding sample change point positons.

The processing device 120 may further designate a body motion sub-signal that meets a duration condition among the multiple body motion sub-signals as the target motion signal. For example, the processing device 120 may determine a body motion sub-signal with the longest duration among the multiple body motion sub-signals as the target motion signal. As another example, the processing device 120 may determine each body motion sub-signal whose duration is greater than a duration threshold as a candidate target motion signal. The processing device 120 may determine the first one or the last one of the candidate target motion signals in chronological order as the target motion signal. Alternatively, the processing device 120 may determine all the candidate target motion signals as the target motion signals.

According to some embodiments, the target motion signal determined by selecting the body motion sub-signal that meets the duration condition can better reflect the true motion situation of the user. Moreover, a relatively stable motion signal can be able to be screened out, thereby further reducing the problem of motion blur artifacts in images caused by body movements.

In some embodiments, the processing device 120 may determine each body motion sub-signal whose average amplitude is less than a preset amplitude threshold as a candidate target motion signal. The processing device 120 may determine a candidate target motion signal with the longest duration among the multiple candidate target motion signals as the target motion signal.

In some embodiments, the preset amplitude threshold may be set according to a default setting of the imaging system 100 or preset by a user or operator via the terminal device 140. In some embodiments, different target objects may correspond to difficult preset amplitude thresholds. The greater the average amplitude of the body motion signal is, the smaller the preset amplitude threshold may be.

In 340, the processing device 120 (e.g., the image generation module 240 illustrated in FIG. 2A) may generate a reconstructed image of the target object by reconstructing the target PET data.

In some embodiments, the processing device 120 may reconstruct the target PET data using an image reconstruction algorithm. The image reconstruction algorithm may include a maximum likelihood expectation method (MLEM), an ordered subsets expectation maximization (OSEM) algorithm, a maximum A posteriori (MAP) algorithm, a mean average precision algorithm, a backward projection algorithm (e.g., a convolution back-projection (CBP) algorithm, a filtering back-projection (FBP) algorithm, a Fourier transform (FT) reconstruction technique, a 3D-rapid prototyping algorithm, or the like, or any combination thereof.

According to some embodiments, the motion signal of the target object determined based on the raw PET data of the target object can reflect a motion situation of the target object during the scanning process more accurately, that is, the motion signal is relatively close to a true motion signal of the target object during the scanning process. Thus, based on the motion signal, target PET data that is less affected by the motion of the target object can be extracted from the raw PET data for reconstruction, thereby reducing or eliminating motion artifacts caused by the motion (e.g., respiratory or body motion) during image reconstruction, and improving the quality of reconstructed images.

In some embodiments, the processing device 120 may further determine a first attenuation coefficient map by processing the reconstructed image e.g., using a second preset model. The processing device 120 may obtain an intermediate reconstructed image by correcting, based on the first attenuation coefficient map, the reconstructed image.

In some embodiments, the processing device 120 may correct the reconstructed image using a second attenuation coefficient map of the target object. For ease of illustration, a CT image is taken as an example of the second attenuation coefficient map in the present disclosure. In some embodiments, the processing device 120 obtains the second attenuation coefficient map of the target object, for example, by scanning the target object using an imaging device or from a storage device.

In some embodiments, since there may be a phase difference between the reconstructed image and the second attenuation coefficient map, during the correcting process, the second attenuation coefficient map may mismatch with the reconstructed image, thus causing attenuation correction artifacts in the attenuation-corrected reconstructed image.

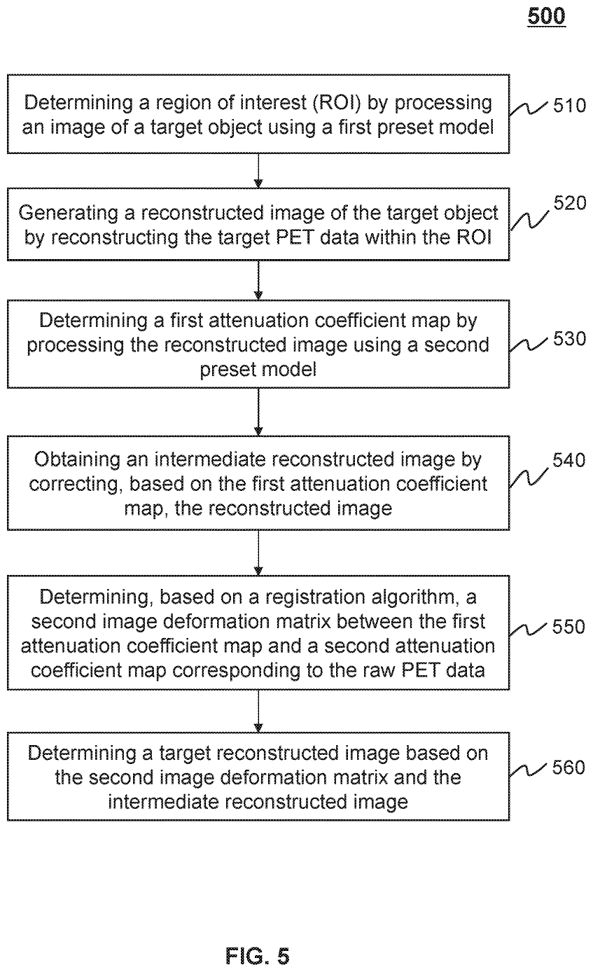

Accordingly, in order to reduce attenuation correction artifacts in the attenuation-corrected reconstructed image, the processing device 120 may further determine a first attenuation coefficient map by processing the reconstructed image using a second preset model. The processing device 120 may obtain an intermediate reconstructed image by correcting, based on the first attenuation coefficient map, the reconstructed image. The processing device 120 may determine, based on a registration algorithm, a second image deformation matrix between the second attenuation coefficient map and the first attenuation coefficient map. The processing device 120 may determine a target reconstructed image based on the second image deformation matrix and the intermediate reconstructed image. More descriptions for determining the target reconstructed image may be found in FIG. 5 and the descriptions thereof.

FIG. 4 is a flowchart illustrating an exemplary process for determining a respiratory motion signal of a target object based on raw PET data of the target object according to some embodiments of the present disclosure. In some embodiments, the process for determining the respiratory motion signal of the target object as described in connection with operation 320 in FIG. 3 may be performed according to the process 400.

In 410, the processing device 120 (e.g., the sample obtaining module 250 illustrated in FIG. 2B) may obtain sample raw PET data of multiple sample target objects. In some embodiments, the processing device 120 may obtain the sample raw PET data from the imaging device 110, the storage device 130, or any other storage device for storage.

In 420, for each of the multiple sample target objects, the processing device 120 (e.g., the sample PET data determination module 260 illustrated in FIG. 2B) may determine sample PET data of a sample ROI of the sample target object based on the sample raw PET data of the sample target object.

The determination manner of the sample ROI of the sample target object may be similar to the determination manner of the ROI of the target object as described in operation 320 in FIG. 3 of the present disclosure.

In 430, the processing device 120 (e.g., the sample respiratory amplitude determination module 270 illustrated in FIG. 2B) may determine, based on the sample PET data, multiple sample true respiratory amplitudes and multiple sample predicted respiratory amplitudes corresponding to the sample target object.

As used herein, the sample true respiratory amplitude refers to a respiratory amplitude of a respiratory motion signal that is close to a true respiratory motion signal of the sample target object. That is, the sample true respiratory amplitude can reflect the true motion situation of the sample target object. The sample predicted respiratory amplitude refers to a respiratory amplitude of a sample respiratory motion signal extracted from the sample raw PET data (also referred to as a sample predicted respiratory motion signal).

The extraction manner of the sample predicted respiratory motion signal from the sample PET data within the sample ROI may be similar to the extraction manner of the respiratory motion signal from the raw PET data within the ROI as described in operation 320 in FIG. 3 of the present disclosure.

For example, the processing device 120 may extract the sample predicted respiratory motion signal using a centroid-based respiratory signal analysis technique. Specifically, the processing device 120 may determine a centroid of the sample ROI. The processing device 120 may determine centroid movement information (i.e., information associated with the movement of the centroid) as the sample predicted respiratory motion signal by processing the sample PET data using a preset algorithm. An amplitude of the sample predicted respiratory motion signal may be represented by a centroid movement distance. In some embodiments, the amplitude of the sample predicted respiratory motion signal may be a true movement amplitude of the centroid. In some embodiments, the amplitude of the sample predicted respiratory motion signal may be a movement amplitude of the centroid projected onto the X-axis, Y-axis, or Z-axis of a three-dimensional cartesian coordinate system.

The processing device 120 may determine, by dividing the sample predicted respiratory motion signal, multiple sample predicted respiratory motion sub-signals (abbreviated as sample predicted sub-signals) under multiple respiratory gates. As used herein, a respiratory gate refers to a strategy to divide a signal. Each respiratory gate corresponds to a sample predicted sub-signal. Specifically, the processing device 120 may divide the sample predicted respiratory motion signal to generate the multiple sample predicted sub-signals using a gating scheme. Each sample predicted sub-signal corresponds to a piece of the sample PET data. The amplitude of the multiple sample predicted sub-signals are different.

In some embodiments, the gating scheme may include an equal amplitude-based gating, an equal count-based gating (CG), a time-based gating (TG), etc. In some embodiments, since if the division of the sample PET data leads to inconsistent counting points of PET data in different respiratory gates, it may result in inconsistent data volumes of the corresponding generated images, which in turn affects the inconsistent noises of the corresponding generated images. Therefore, in order to make the noise of each gated image the same, the processing device 120 may preferably adopt the equal count-based gating, so as to ensure that the noise of each gated image is the same.

Further, the processing device 120 may determine, based on the multiple sample predicted sub-signals, the multiple sample true respiratory amplitudes and the multiple sample predicted respiratory amplitudes corresponding to the sample target object.

In some embodiments, for each of the multiple respiratory gates, the processing device 120 determines a difference between a maximum respiratory amplitude and a minimum respiratory amplitude of a sample predicted sub-signal as the sample predicted respiratory amplitude corresponding to the respiratory gate.

In some embodiments, for each of the multiple respiratory gates, the processing device 120 may determine an average respiratory amplitude of the sample predicted sub-signal corresponding to the respiratory gate. The processing device 120 may determine a sample predicted respiratory amplitude corresponding to the respiratory gate by differentiating the average respiratory amplitude corresponding to the respiratory gate with an average respiratory amplitude corresponding to any other respiratory gate of the multiple respiratory gates.

Merely by way of example, the processing device 120 may determine a minimum average respiratory amplitude among the average respiratory amplitudes corresponding to the multiple respiratory gates. The processing device 120 may determine the sample predicted respiratory amplitude corresponding to the respiratory gate by differentiating the average respiratory amplitude corresponding to the respiratory gate with the minimum average respiratory amplitude. For example, among N sample predicted sub-signals, the sample predicted sub-signal with the minimum average respiratory amplitude may be subtracted from the other N-1 sample predicted sub-signals to obtain N-1 amplitude differences, i.e., N-1 sample predicted respiratory amplitudes. Meanwhile, for the sample predicted sub-signal with the minimum average respiratory amplitude, the corresponding sample predicted respiratory amplitude may be determined as 0 by substracting it from itself.

In some embodiments, for each of the multiple respiratory gates, the processing device 120 may determine an amplitude difference between the average respiratory amplitude corresponding to the respiratory gate and an average respiratory amplitude corresponding to each of the other respiratory gates of the multiple respiratory gates. The processing device 120 may determine an average value of the multiple amplitude differences corresponding to the other respiratory gates of the multiple respiratory gates as the sample predicted respiratory amplitude corresponding to the respiratory gate.

In some embodiments, the multiple sample predicted respiratory amplitudes may be normalized.

According to some embodiments of the present disclosure, by determining a relative respiratory amplitude corresponding to each respiratory gate as the sample predicted respiratory amplitude of the sample target object, the sample predicted respiratory amplitude corresponding to that respiratory gate can accurately be characterized.

For each of the multiple respiratory gates, the processing device 120 may generate a PET sub-image by reconstructing sample PET sub-data corresponding to a sample predicted sub-signal corresponding to the respiratory gate. In some embodiments, the PET sub-image corresponding to each respiratory gate may be a PET sub-image before or after attenuation correction. In some embodiments, the PET sub-image corresponding to each respiratory gate may be a PET direct back projection image (histoimage).

In some embodiments, the processing device 120 may determine a first image deformation matrix corresponding to the respiratory gate by registering the PET sub-image corresponding to the respiratory gate with a PET sub-image corresponding to any other respiratory gate of the multiple respiratory gates. Merely by way of example, the processing device 120 may determine the first image deformation matrix corresponding to the respiratory gate by registering the PET sub-image with a PET sub-image corresponding to the minimum average respiratory amplitude using a registration algorithm. In some embodiments, the registration algorithm may include a rigid transformation-based registration algorithm, an affine transformation-based registration algorithm, B-spline registration algorithm, an optical flow-based registration algorithm, a deep learning-based registration algorithm, or the like, or any combination thereof.

The first image deformation matrix may be a three-dimensional matrix with a size same as a gating area of the corresponding respiratory gate (e.g., an area of the PET sub-image corresponding to the respiratory gate). The first image deformation matrix may represent deformation parameters of a whole scanning region of the PET sub-image.

The processing device 120 may determine, based on a mean value of the first image deformation matrix within the sample ROI, a sample true respiratory amplitude corresponding to the respiratory gate. The first image deformation matrix may represent the motion of each point (e.g., a pixel or voxel) in the PET sub-image corresponding to the respiratory gate or the PET sub-image corresponding to any other respiratory gate of the multiple respiratory gates along the X-axis, Y-axis, or Z-axis of the three-dimensional cartesian coordinate system. Thus, in some embodiments, the mean value of the first image deformation matrix within the sample ROI may be a whole mean value of the first image deformation matrix or a mean value of the first image deformation matrix along a specific direction.

According to some embodiments of the present disclosure, by registering to determine the first image deformation matrix corresponding to each respiratory gate, and then calculating the mean value of the first image deformation matrix in the respiratory affected area (i.e., the sample ROI), a more accurate sample true respiratory amplitude can be obtained.

In some embodiments, the processing device 120 may determine the sample true respiratory amplitude corresponding to the respiratory gate using a respiratory amplitude determination model. The respiratory amplitude determination model may be a machine learning model trained based on a plurality of groups of training data. Each group of training data includes a sample PET sub-image, and during the training, the label is the corresponding sample true respiratory amplitude.

In 440, the processing device 120 (e.g., the correction factor determination module 290 illustrated in FIG. 2B) may determine a sample correction factor by performing a regression analysis on the multiple sample true respiratory amplitudes and the multiple sample predicted respiratory amplitudes corresponding to the sample target object.

In some embodiments, the regression analysis may include a polynomial regression analysis technique, a linear regression analysis technique, or other regression analysis techniques, as well as machine learning or deep learning based analysis techniques. Specifically, the processing device 120 may determine a slope of each sample true respiratory amplitude relative to the corresponding sample predicted respiratory amplitude as the sample correction factor. Further, the processing device 120 may determine multiple sample correction factors corresponding to the multiple sample target object.

In some embodiments, for each of the multiple sample target objects, the processing device 120 may determine a maximum sample predicted respiratory amplitude and a minimum sample predicted respiratory amplitude among the sample predicted respiratory amplitudes. Further, the processing device 120 may determine a maximum sample true respiratory amplitude and a minimum sample true respiratory amplitude among the sample true respiratory amplitudes. The processing device 120 determines a first difference between the maximum sample predicted respiratory amplitude and the minimum sample predicted respiratory amplitude, and a second difference between the maximum sample true respiratory amplitude and the minimum sample true respiratory amplitude. The processing device 120 determines a ratio of the second difference to the first difference as the sample correction factor.

In 450, the processing device 120 (e.g., the correction factor determination module 290 illustrated in FIG. 2B) may determine the correction factor based on multiple sample correction factors corresponding to the multiple sample target objects. For example, the processing device 120 may determine an average value of the multiple sample correction factors corresponding to the multiple sample target objects as the correction factor. As another example, the processing device 120 may determine a median value of the multiple sample correction factors corresponding to the multiple sample target objects as the correction factor.

According to some embodiments of the present disclosure, by determining the correction factor and correcting the respiratory motion signal extracted from raw PET data of a target object based on the correction factor, the corrected respiratory motion signal can be closer to the true respiratory motion signal of the target object during the scanning process.

FIG. 5 is a flowchart illustrating an exemplary process for determining a target reconstructed image according to some embodiments of the present disclosure. In some embodiments, the process for determining the target reconstructed image as described in connection with operation 340 in FIG. 3 may be performed according to the process 500.

In 510, the processing device 120 (e.g., the image generation module 240 illustrated in FIG. 2A) may determine a region of interest (ROI) by processing an image of a target object using a first preset model.

The processing device 120 may determine the ROI in connection with operation 320 in FIG. 3. For ease of illustration, a CT image is taken as an example of the image in the present disclosure.

In 520, the processing device 120 (e.g., the image generation module 240 illustrated in FIG. 2A) may generate a reconstructed image of the target object by reconstructing the target PET data within the ROI.

The processing device 120 may generate the reconstructed image (i.e., a PET image) in connection with operation 340 in FIG. 3.

Since the target object may be in different motion situations when scanning the target object to generate the PET image (i.e., the reconstructed image) and a second attenuation coefficient map (e.g., a CT image), there may be a phase difference between the CT image and the PET image. Thus, if the second attenuation coefficient map is directly used for correcting the reconstructed image, the reconstructed image may include attenuation correction artifacts, thereby decreasing the image quality.

In 530, the processing device 120 (e.g., the image generation module 240 illustrated in FIG. 2A) may determine a first attenuation coefficient map by processing the reconstructed image using a second preset model.

The first attenuation coefficient map and the second attenuation coefficient map are of the same type of image. Thus, when the second attenuation coefficient map is a CT image, the first attenuation coefficient map is also a CT image. Exemplarily, the second attenuation coefficient map is a first CT image, and the first attenuation coefficient map is a second CT image.

The processing device 120 may input the reconstructed image into the second preset model to determine the first attenuation coefficient map. The second preset model may be a deep learning network trained based on a plurality of groups of training data. Each group of training data includes a sample reconstructed image (i.e., a sample PET image), and during the training, the label is a sample first attenuation coefficient map (e.g., a sample CT image) registered with the sample PET image. In other words, in each group of training data, there is no phase difference between the sample first attenuation coefficient map and the sample PET image. As a result, there is no phase difference between the first attenuation coefficient map (i.e., the second CT image) and the reconstructed image (i.e., the PET image).

In some embodiments, the second preset model may include a convolutional neural network (CNN) model, a generative adversarial network (GAN) model, a fully convolutional neural network (FCN) (e.g., a U-Net, a V-Net), a recurrent neural network (RNN), a diffusion model, a transformer, or the like, or any combination thereof.

In 540, the processing device 120 (e.g., the image generation module 240 illustrated in FIG. 2A) may obtain an intermediate reconstructed image by correcting, based on the first attenuation coefficient map, the reconstructed image.

Since the first attenuation coefficient map is aligned with the reconstructed image, the intermediate reconstructed image (i.e., an intermediate PET image) is aligned with the first attenuation coefficient map.

In 550, the processing device 120 (e.g., the image generation module 240 illustrated in FIG. 2A) may determine, based on a registration algorithm, a second image deformation matrix between the second attenuation coefficient map and the first attenuation coefficient map. The second image deformation matrix is a matrix composed of deformation parameters of the first attenuation coefficient map relative to the second attenuation coefficient map.

In 560, the processing device 120 (e.g., the image generation module 240) may determine a target reconstructed image based on the second image deformation matrix and the intermediate reconstructed image.

The processing device 120 may apply the second image deformation matrix in the intermediate reconstructed image to determine the target reconstructed image (i.e., a target PET image). There is no phase difference between the second attenuation coefficient map and the target reconstructed image. That is, the target reconstructed image may be aligned with the second attenuation coefficient map. Thus, the second attenuation coefficient map can be used to correct the target reconstructed image directly.

According to some embodiments of the present disclosure, since the intermediate reconstructed image is aligned with the first attenuation coefficient map, after applying the second image deformation matrix in the intermediate reconstructed image, the obtained target reconstructed image may be aligned with the second attenuation coefficient map, thus resolve the problem of image mismatch in PET/CT image fusion caused by the phase difference between the CT image and the PET image.

The operations of the illustrated processes 300, 400, and 500 presented above are intended to be illustrative. In some embodiments, a process may be accomplished with one or more additional operations not described, and/or without one or more of the operations discussed. Additionally, the order in which the operations of a process described above is not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer-readable media having computer-readable program code embodied thereon.

A non-transitory computer-readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer-readable signal medium may be any computer-readable medium that is not a computer-readable storage medium and that may communicate, propagate, or transport a program for use by or in connection with an instruction execution system, apparatus, or device. Program code embodied on a computer-readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object-oriented programming language such as Java, Scala, Smalltalk, Eiffel, JADE, Emerald, C++, C #, VB. NET, Python or the like, conventional procedural programming languages, such as the “C” programming language, Visual Basic, Fortran, Perl, COBOL, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof to streamline the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A system for image reconstruction, comprising:
at least one storage device storing executable instructions; and
at least one processor in communication with the at least one storage device, wherein when executing the executable instructions, the at least one processor is configured to cause the system to perform operations including:
obtaining raw positron emission tomography (PET) data of a target object;
determining a motion signal of the target object based on the raw PET data of the target object;
extracting, based on the motion signal, target PET data from the raw PET data, the target PET data corresponding to a target motion signal, in the motion signal, in which motion change of the target object meets a predetermined change condition; and
generating a reconstructed image of the target object by reconstructing the target PET data.

2. The system of claim 1, wherein the determining a motion signal of the target object based on the raw PET data of the target object comprises:
determining a mask of a region of interest (ROI) by processing an image of the target object, and
determining, based on the mask of the ROI, the motion signal from the raw PET data within the ROI.

3. The system of claim 2, wherein the determining a motion signal of the target object based on the raw PET data of the target object comprises:
determining a centroid of the ROI;
determining centroid movement information of the centroid by processing the raw PET data; and
determining the motion signal based on the centroid movement information of the centroid, wherein an amplitude of the motion signal is represented by a centroid movement distance of the centroid.

4. The system of claim 1, wherein the motion signal includes a respiratory motion signal, and the determining a motion signal of the target object based on the raw PET data of the target object further comprises:
determining a correction factor; and
correcting the motion signal based on the correction factor.

5. The system of claim 4, wherein the determining a correction factor comprises:
obtaining sample raw PET data of multiple sample target objects;
for each of the multiple sample target objects,
determining sample PET data within a sample ROI of the sample target object based on the sample raw PET data of the sample target object;
determining, based on the sample PET data, multiple sample true respiratory amplitudes and multiple sample predicted respiratory amplitudes corresponding to the sample target object;
determining a sample correction factor by performing a regression analysis on the multiple sample true respiratory amplitudes and the multiple sample predicted respiratory amplitudes corresponding to the sample target object; and
determining the correction factor based on multiple sample correction factors corresponding to the multiple sample target objects.

6. The system of claim 5, wherein the determining, based on the sample PET data, multiple sample true respiratory amplitudes and multiple sample predicted respiratory amplitudes corresponding to the sample target object comprises:
determining centroid movement information as a sample predicted respiratory motion signal by processing the sample PET data using the preset algorithm, wherein an amplitude of the sample predicted respiratory motion signal is represented by a centroid movement distance;
determining, by dividing the sample predicted respiratory motion signal, multiple sample predicted sub-signals under multiple respiratory gates; and
determining, based on the multiple sample predicted sub-signals, the multiple sample true respiratory amplitudes and the multiple sample predicted respiratory amplitudes corresponding to the sample target object.

7. The system of claim 6, wherein the determining, based on the multiple sample predicted sub-signals, the multiple sample predicted respiratory amplitudes corresponding to the sample target object comprises:
for each of the multiple respiratory gates,
determining an average respiratory amplitude based on a sample predicted sub-signal corresponding to the respiratory gate; and
determining a sample predicted respiratory amplitude corresponding to the respiratory gate by differentiating the average respiratory amplitude corresponding to the respiratory gate with an average respiratory amplitude corresponding to any other respiratory gate of the multiple respiratory gates.

8. The system of claim 6, wherein the determining, based on the multiple sample predicted sub-signals, the multiple sample true respiratory amplitudes corresponding to the sample target object comprises:
for each of the multiple respiratory gates,
generating a PET sub-image by reconstructing sample target PET sub-data corresponding to a sample predicted sub-signal corresponding to the respiratory gate;
determining a first image deformation matrix corresponding to the respiratory gate by registering the PET sub-image with a PET sub-image corresponding to any other respiratory gate of the multiple respiratory gates; and
determining, based on a mean value of the first image deformation matrix within the sample ROI, a sample true respiratory amplitude corresponding to the respiratory gate.

9. The system of claim 4, wherein the motion signal includes the respiratory motion signal, and the target motion signal is determined by:
selecting, from the respiratory motion signal, a candidate motion signal whose respiratory amplitude is less than or equal to an amplitude threshold; and
determining the target motion signal based on the candidate motion signal.

10. The system of claim 9, wherein the determining the target motion signal based on the candidate motion signal comprises:
selecting, from the candidate motion signal, multiple consecutive respiratory motion sub-signals in an amplitude direction; and
designating a consecutive respiratory motion sub-signal that meets an amplitude span requirement among the multiple consecutive respiratory motion sub-signals as the target motion signal.

11. The system of claim 1, wherein the motion signal includes a body motion signal, and the target motion signal is determined by:
determining one or more change point positions by processing the motion signal;
determining multiple body motion sub-signals by dividing, based on the one or more change point positions, the motion signal; and
designating a body motion sub-signal that meets a duration condition among the multiple body motion sub-signals as the target motion signal.

12. The system of claim 1, wherein the operations further include:
determining a first attenuation coefficient map by processing the reconstructed image using a second preset model; and
obtaining an intermediate reconstructed image by correcting, based on the first attenuation coefficient map, the reconstructed image.

13. The system of claim 12, wherein the operations further include:
obtaining a second attenuation coefficient map of the target object, the second attenuation coefficient map corresponding to the raw PET data;
determining a second image deformation matrix between the second attenuation coefficient map and the first attenuation coefficient map; and
determining a target reconstructed image based on the second image deformation matrix and the intermediate reconstructed image.

14. A method for image reconstruction, implemented on a computing device having at least one processor and at least one storage device, the method comprising:
obtaining raw positron emission tomography (PET) data of a target object;
determining a motion signal of the target object based on the raw PET data of the target object;
extracting, based on the motion signal, target PET data from the raw PET data, the target PET data corresponding to a target motion signal, in the motion signal, in which motion change of the target object meets a predetermined change condition; and
generating a reconstructed image of the target object by reconstructing the target PET data.

15. The method of claim 14, wherein the determining a motion signal of the target object based on the raw PET data of the target object comprises:
determining a mask of a region of interest (ROI) by processing an image of the target object, and
determining, based on the mask of the ROI, the motion signal from the raw PET data within the ROI.

16. The method of claim 15, wherein the determining a motion signal of the target object based on the raw PET data of the target object comprises:
determining a centroid of the ROI;
determining centroid movement information of the centroid by processing the raw PET data; and
determining the motion signal based on the centroid movement information of the centroid, wherein an amplitude of the motion signal is represented by a centroid movement distance of the centroid.

17. The method of claim 14, wherein the motion signal includes a respiratory motion signal, and the determining a motion signal of the target object based on the raw PET data of the target object further comprises:
determining a correction factor; and
correcting the motion signal based on the correction factor.

18. The method of claim 14, wherein the determining a correction factor comprises:
obtaining sample raw PET data of multiple sample target objects;
for each of the multiple sample target objects,
determining sample PET data within a sample ROI of the sample target object based on the sample raw PET data of the sample target object;
determining, based on the sample PET data, multiple sample true respiratory amplitudes and multiple sample predicted respiratory amplitudes corresponding to the sample target object;
determining a sample correction factor by performing a regression analysis on the multiple sample true respiratory amplitudes and the multiple sample predicted respiratory amplitudes corresponding to the sample target object; and
determining the correction factor based on multiple sample correction factors corresponding to the multiple sample target objects.

19. A system for image reconstruction, comprising:
at least one storage device storing executable instructions; and
at least one processor in communication with the at least one storage device, wherein when executing the executable instructions, the at least one processor is configured to cause the system to perform operations including:
obtaining raw positron emission tomography (PET) data of a target object;
determining a respiratory motion signal of the target object based on the raw PET data of the target object;
correcting the respiratory motion signal based on a correction factor, wherein the correction factor is determined based on sample respiratory motion signals obtained using two different manners; and
generating a reconstructed image of the target object based on the corrected respiratory motion signal and the raw PET data.

20. The system of claim 19, wherein the two different manners at least include:
a first manner of extracting the sample respiratory motion signals from sample raw PET data, and
a second manner of extracting the sample respiratory motion signals from reconstructed PET sub-images corresponding to sample PET sub-data, wherein the sample PET sub-data is generated by gating the sample raw PET data.
